(19) United States
(12) Patent Application Publication
(10) Pub. No.: US 2011/0207158 A1
(43) Pub. Date: Aug. 25, 2011

(54) Method for the non-invasive detection of microorganisms in a closed container

(75) Inventor: Jacobus Johannes Frederik Van Veen, Badhoevedorp (NL)

(73) Assignee: Nederlandse Organisatie voor Toegepast-natuurwetenschappelijk onderzoek TNO, Delft (NL)

(21) Appl. No.: 10/527,121

(22) PCT Filed: Sep. 10, 2003
(86) PCT No.: PCT/NL03/00629

§ 371 Date: Sep. 28, 2005

(30) Foreign Application Priority Data
Sep. 10, 2002 (NL) 1021420

(51) Int. Cl. C12Q 1/02; C12Q 1/40; C12Q 1/37; C12Q 1/44; C12Q 1/34; C12M 1/40

(52) U.S. Cl. 435/19; 435/29; 435/22; 435/23; 435/18; 435/288.7

(57) ABSTRACT

The present invention relates to a method for detecting a contamination with a microorganism in a closed container, in which an extracellular enzyme of the microorganism is detected. The method can very suitably be carried out by providing a substrate for this enzyme to the contents of the container and detecting the conversion of this substrate by this enzyme. An embodiment of an apparatus according to the invention is shown in, which shows a container (1) for, for instance, sterile tissue culture of plants (2). The container is provided with a growth medium (3) which comprises a substrate for a microbial extracellular enzyme and with optical sensors (4) which can be read by means of an optical measuring device (5). An alternative embodiment is shown which shows a sterile package for, for instance, medical aids (2). The package forms a closed container (1) whose wall is provided with an indicator (3) which can be visually read.

The invention relates to a method for the non-invasive detection of a contamination with a microorganism in a closed container. The invention specifically relates to a method for detecting extracellular enzyme activity of a microorganism in a closed container.

In most cases, contamination of a product with microorganisms, such as a contamination of a (sterile) tissue culture with bacteria, results in loss of the product. Since particular germs of microorganisms may reside in a raw material and/or my find their way into the product during the production process, it is conventional that raw materials and/or end products are subjected to a sterilization, preferably at a latest possible time in the production process. This sterilization is carried out for the purpose of killing the germs (possibly) present and can, for instance, consist in a heat treatment, a chemical treatment or a radiation treatment.

To preclude contamination of a sterilized product as much as possible, these products are preferably kept in a closed container, or surrounded by a barrier impenetrable to microorganisms.

In most uses related to closed and sterile containers, as is, for instance, the case in sterile tissue culture, it is important that there is certainty whether or not microorganisms are present.

However, conventional methods for detecting microorganisms in a food product or a clinical sample in a closed container or surrounded by a barrier require that the container or the barrier be briefly opened, that a sample be taken and that this sample be checked for the presence of microorganisms. However, each opening of the container or the barrier, however brief, increases the risk of contamination.

In many cases, it can only be determined afterwards and after opening the container whether the container was sterile. Only in those cases where there is a very large degree of contamination, can it possibly be visually determined whether the container is contaminated without opening it.

Another disadvantage of the necessity to open a container for determining its sterility relates to the danger of spread and growth of a contamination present in the container. Here, it is highly important that a contaminated container is not further used or, if possible, is not even opened. This plays a role in, for instance, sterile tissue culture, where the medium has to be changed regularly, or in a sterile culture of plant tissue, where, after an initial growth of a few weeks, the plants have to be planted out. The possibility to determine a contamination in the container without having to open it contributes to the increase of the quality and efficiency of the operations.

U.S. Pat. No. 6,197,577 describes the use of a sensor for the detection of microorganisms that does not require a container to be opened (non-invasive method), consisting of a (specific) growth medium present as a layer on a sensor (indicator layer). However, a disadvantage of this method is that the microorganism can only be detected locally, at the location of the sensor. If the microorganism and the sensor are not in direct contact with each other, detection of the presence of the microorganism is not possible.

It has now been found that a contamination with a microorganism in a closed container can be detected by detecting the presence of an extracellular enzyme of a microorganism in the container. The detection of an extracellular enzyme can, for instance, very suitably take place by means of adding a substrate of this extracellular enzyme to the container and the detection of the conversion of this substrate by this extracellular enzyme by means of a sensor. Such an added substrate can, for instance, be homogeneously distributed through the contents of the container or optionally be applied as a coating on the inner wall of the container.

The present invention solves the problem of the prior art methods for the detection of microorganisms.

The present invention provides a method for detecting a contamination with a microorganism in a closed container, in which an extracellular enzyme of this microorganism is detected. By detecting the extracellular enzymes of the microorganism or detecting a specific conversion product, the location of the microorganism itself and that of the indicator or sensor are less limited.

In principle, any contamination with a microorganism can be detected by means of a method according to the invention. The present invention is especially suitable for uses in which a contamination in a closed container needs to be detected without needing to open the container for this purpose. Thus, the invention provides a non-invasive method for detecting a contamination with a microorganism.

Microorganisms which can be detected by use of a method according to the invention are particularly microorganisms which produce extracellular enzymes. Such a production of extracellular enzymes is known in plant cells, protozoa, fungi, yeasts, archaea and bacteria. An extracellular enzyme is an enzyme which is secreted by the organism from the cell into the surrounding medium (exoenzyme). So, all these microorganisms can be detected by means of a method of the invention. Depending on the choice of the added substrate, groups of microorganisms can be detected, for instance bacteria or fungi, and possibly even virtually the whole group of microorganisms can be detected. In the latter case, a generic method for demonstrating the presence of microorganisms is involved. Preferably, fungi, yeasts and bacteria are detected as a group. There is a particular preference for the detection of bacteria.

The detection of an extracellular enzyme of a microorganism (whose production can optionally be induced by adding an inducing substance to the container, which substance will, in many cases, be the substrate mentioned) can, for instance, take place by demonstrating the enzyme itself.

Extracellular enzymes produced by microorganisms which can be detected by means of a method of the invention are enzymes which are freely present in the medium surrounding the organism and thus not membrane-bound, such as amylase (e.g. alpha-amylase), protease (e.g. gelatinase, caseinase and elastase), lipase, peroxidase, catalase, alcohol dehydrogenase, pectinase, xylanase, cellulase, chitinase, collagenase, hyaluronidase, phospholipase (e.g. lecithinase) and glucanase (e.g. β-glucanase); preferably an organic polymer-hydrolyzing extracellular enzyme, such as an amylase, protease, lipase, pectinase, xylanase, cellulase, chitinase, collagenase or glucanase; more preferably an amylase or protease, and even more preferably a gelatinase or caseinase. By means of the detection of the presence of at least one or a combination of such extracellular enzymes, a microorganism which produces these extracellular enzymes can be detected. So, by inducing extracellular enzyme activity of a microorganism, a method for generic detection of microorganisms can be provided.

The demonstration of an extracellular enzyme can be done in a manner known to a person skilled in the art, for instance by means of methods for detection of proteins (such as enzymes). For instance, an immunoassay can be used for detecting an extracellular enzyme, but also other methods, such as the use of lectins, are suitable. Preferably, this direct form of detection is specific for a particular extracellular enzyme and is based on an optical change (absorption, fluorescence, and the like), enabling a non-invasive measurement.

It is also possible to detect not the enzyme itself, but its activity. An example of this is the detection of a conversion of, for instance, a fluorogenic substrate into a product, via a reaction catalyzed by the extracellular enzyme.

In particular embodiments of the invention, it will not be necessary to add a substrate for an extracellular enzyme to the container. Such a substrate can already be present in the container, for instance as a component of the growth medium.

In a preferred embodiment, before use, a substrate for an extracellular enzyme is added to the container for the purpose of determining its activity. This added substrate is preferably provided so as to be distributed as homogeneously as possible through the container. In such an embodiment, the substrate can, for instance, be provided in a growth medium or optionally be applied as a coating on the inner wall of the container.

Preferably, detection of an extracellular enzyme takes place by its reaction with a substrate present in a sensor layer (indicator layer) as described hereinbelow. This substrate can be a natural or a synthetic substrate for the extracellular enzyme. In that case, the substrate is preferably labeled, for instance with a dye or a fluorescence indicator to detect the presence of the extracellular enzyme.

More preferably, a method according to the invention comprises the detection of the conversion of the quantity of a substrate and/or the detection of a reaction product of a reaction catalyzed by an extracellular enzyme.

The choice of the extracellular enzyme and/or reaction product is preferably made such that these can only come from a microorganism, and, for instance, not from a tissue in a tissue culture. The choice of the substrate is preferably made such that it can only be converted by the microorganism. For instance, a microorganism which takes up the substrate and converts it into a product can be detected by detection of the product formed and secreted or by detection of the quantity of substrate taken up and consumed. In fact, any microorganism-specific substrate conversion be used for the detection of a microorganism in a method according to the invention. Substrates which can be converted by large groups of microorganisms are preferred, giving the method a generic character.

Suitable substrates which can be used in embodiments of the invention are, for instance, starch, particular proteins, glyoxylate, substances with an aldehyde function, carboxyl esters or acetic acid esters and particular vitamins, preferably a protein, such as BSA, casein, whey proteins, gelatin, etc. Preferably, a substrate or a combination of substrates is added to the container for the purpose of inducing the production of an extracellular enzyme to be detected of the microorganism, but a substrate can, for instance, also be used to detect the enzyme.

As mentioned hereinabove, it is possible to label a substrate, as, for instance, present in the sensor layer or, for instance, homogeneously distributed through the container, in order to determine its conversion by an extracellular enzyme of a microorganism. Very suitable labels are, for instance, labels which produce a fluorescent, color (chromogenic) or light signal (luminescent) and with which the conversion of the substrate can be detected optically and thus non-invasively. Here, the label can, for instance, be chosen such that the conversion of the substrate results in the separation of the label or that the conversion effects an optical change in the label.

It is also possible to detect a substrate and/or reaction product by means of an indicator. For instance, iodine can be used as an indicator to detect the decomposition of starch resulting from the presence of amylase in a closed container. So, the occurrence of a reaction of an extracellular enzyme with a substrate can be detected by direct detection of the converted substrate, for instance because it changes color or obtains a (changing) fluorescent character, but also indirectly, for instance by additionally adding an indicator substance, such as, for instance, iodine.

Such an indicator can also be formed by, for instance, crystal violet lactones which are specific for alcohols, Amplex Red for the detection of peroxides, bromothymol blue in a suitable matrix for the detection of ammonia, or an O₂ or pH indicator if these parameters are involved in the enzymatic reaction. Known oxygen indicators are fluorescent ruthenium complexes, known pH indicators are, for instance, phenol red, (bromo)thymol blue, congo red, cresol red, etc. These indicators can be very suitably immobilized in a polymer matrix, either covalently bound or captured.

Indicators can be added to the whole or to a part of the contents of the container. Preferably, an indicator is added to the container by incorporating the indicator in a material, for instance on a polymer basis, which can be attached on the inner side of the container. For instance, an indicator can be incorporated in a coating located on the inner side of this container, either over the whole surface of the container or locally, in the form of a kind of ‘sticker’.

The invention is highly advantageous for uses in which the container is preferably not opened, because contamination can then already be detected in an early stage and measures can be taken timely (such as removal of the contaminated container and/or starting up a new production process which can replace the contaminated product).

Therefore, to detect an extracellular enzyme or a substrate or a reaction product thereof, preferably a measurement is used in which the container is not opened. As said, for this purpose, an optical measurement can very suitably be used. Preferably, an optical measurement is used in which the measurement takes place through the wall of the container. For this purpose, at least a part of the wall of the container needs to be transparent.

As said, an optical measurement for the detection of an extracellular enzyme, a substrate thereof or a reaction product of a conversion thereof can, for instance, comprise the measurement of a fluorescent, color or (chemi)luminescent light signal. For this purpose, for instance, a substrate of an extracellular enzyme can be labeled fluorescently or (chemi)luminescently, which provides a fluorescent or (chemi)luminescent signal upon conversion. Then, this light signal can be measured by means of an optical measuring device suitable for this purpose, or by means of visual inspection. An important advantage of an instrumental detection is that it allows the method to be automated and a human factor is no longer required.

A preferred embodiment according to the invention makes use of a sensor system, more preferably an optical sensor, by means of which ah optochemical measurement can be carried out in which it is provided on at least a part of the inner side of the container.

An alternative embodiment relates to a sensor reflecting the accumulative enzyme activity, i.e. a sensor for time-integrated detection, comparable to a dosimeter. This has the advantage that information about a contamination is also obtained if the measurement is carried out or read at the moment that the microorganisms have already died. Such a sensor can, for instance, comprise a fluorescein or sulforhodamine-labeled casein fluorescent indicator and is particularly suitable in uses for the sterile (tissue) culture of plants, in which case such a sensor is thus preferably used.

Optochemical sensors are known to a person skilled in the art. For instance, optochemical sensors as described in U.S. Pat. No. 5,541,113, U.S. Pat. No. 5,611,998, U.S. Pat. No. 5,866,433, EP 1 199 556, U.S. Pat. No. 6,254,829 or WO 01/69243 can be used.

In another aspect, the invention provides a container for sterile tissue culture which comprises an indicator or an optochemical sensor function for detecting an extracellular enzyme of a microorganism.

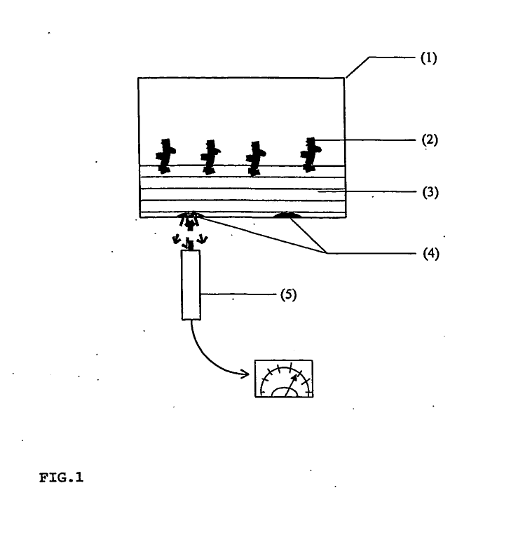

FIG. 1 diagrammatically shows an example of a container according to the invention such as it can be used in sterile tissue culture of plants, with sensors for instrumental reading provided in the bottom of the container. The Figure shows the container (1) for sterile tissue culture of plants (2), which is provided with a growth medium (3) comprising a substrate for a microbial extracellular enzyme and with optical sensors (4) which can be read by means of an optical measuring device (5).

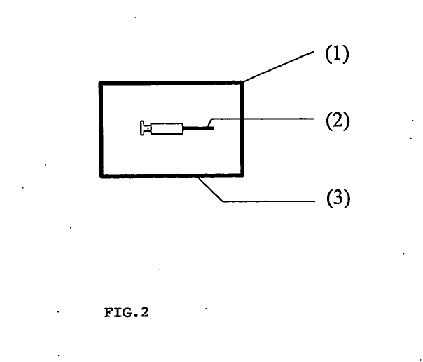

FIG. 2 diagrammatically shows an example of a container according to the invention such as it can be used in sterile packages for medical or paramedical products with visual reading, with the whole inner side of the container wall (in this case, the package) being coated with an indicator layer. The Figure shows a sterile package with a medical aid (2), which package is formed by a closed container (1) whose wall is provided with an indicator (3) which can be visually read.

The method and container according to the invention can exceedingly well be used in tissue culture procedures, in which it is important that the container in which the tissue is present is not opened anymore during the culture, and in which detection of contamination is desired.

Methods and containers according to the invention can exceedingly well be used in sterilely packaged medicines, sterilely packaged medical aids, such as syringes or surgical instruments, sterilely packaged sticking plasters and other medical packages.

The invention can also be used for the detection of, for instance, cell lysis of cultured material in a tissue culture. In this case, enzymes of the tissue culture product can be detected by use of a method according to the invention when they are released into the medium due to lysis. In this manner, the quality of the product can be monitored.

The invention will now be illustrated on the basis of the following examples, which are not to be construed to be limitative.

### EXAMPLE 1

### Sterile (Tissue) Culture of Plants

A container for sterile tissue culture of plants is manufactured according to a method familiar to a person skilled in the art. In this, the substrate is, for instance, formed by a rockwool block saturated with a (liquid) medium (e.g. Murashige and Skoog medium) to serve as a nutrient medium for the plant. Then, to the rockwool block, globules grafted with plant tissue or, for instance, pieces of tissue of the plant are added in a manner known to a person skilled in the art. The whole is packaged in a completely enclosing manner in an aseptic, breathable foil impermeable to microorganisms. All components, with the exception of the living plant tissue, are sterilized in advance and the whole is packaged under sterile conditions, for instance in a flow cabinet.

After a period of 4 to 6 weeks, the plants need to be planted out and the foil bags (containers) are cut open. This can optionally take place completely automatically.

Prior to the moment of cutting open the containers, it is determined whether or not the respective container is contaminated with a microorganism. This is done by use of a method according to the invention. For this purpose, prior to adding the medium to the rockwool substrate or optionally after this, a substrate of an extracellular enzyme is added to the medium as described hereinabove, optionally in combination with an indicator substance, so that either a specific coloring of the medium or an optical change in a sensor located on the inner wall of the foil can be observed. A few specific examples of this elaboration are elaborated hereinbelow.

### EXAMPLE 2

### Addition of Starch and Detection of α-Amylase-Producing Microorganisms

To the medium as used in Example 1, approximately 1.0 wt. % of starch is added. This induces α-amylases (EC 3.2.1.1.) (in bacteria, fungi as well as yeasts) which convert the starch into polysaccharide fragments. To the added starch, a dye is bound which is separated upon a reaction between the α-amylase and the starch and is released into the medium. When enzyme activity is sufficient, the medium will color as a result of this. This coloring is determined by means of an optical inspection system or visually.

### EXAMPLE 3

### Addition of Starch and Detection of α-Amylase-Producing Microorganisms by Means of a Sensor

This example is carried out in the same manner as Example 2, with the difference that, here, an addition of starch without a dye being bound thereto is involved. Instead, the presence of α-amylases is demonstrated by means of a sensor (a kind of sticker) located on the inner wall of the foil. This sensor comprises a fluorescently labeled substrate which is converted by α-amylases. In this conversion, the fluorescence properties change, which is determined using an optical reading unit. In this case, the production of amylases will take place in the whole medium, but their detection takes place locally.

### EXAMPLE 4

### Addition of Riboflavin

In this example, riboflavin is added to the medium of Example 1 in a quantity between, for instance, 1-100 ppm. This induces riboflavinases (in any case with many bacteria) which convert riboflavin into ribitol and lumichrome. The detection method is comparable to that of Example 2 or 3: lumichrome is a fluorescent substance which can be demonstrated in the medium itself by means of an inspection system as set forth in Example 2, but the detection of the presence of riboflavinase can also take place in a sensor (locally) as set forth in Example 3.

### EXAMPLE 5

### Addition of Protein

In this example, a quantity of protein (0.05-1 wt. %) is added to the medium of Example 1. Preferably, this quantity is as low as possible because of the costs. This induces extracellular proteases in the microorganisms tested, which are detected by a sensor/indicator reflecting either the actual protease activity or the time-integrated (accumulative) protease activity. Eligible proteins include BSA (bovine serum albumin), casein, whey proteins and gelatin.

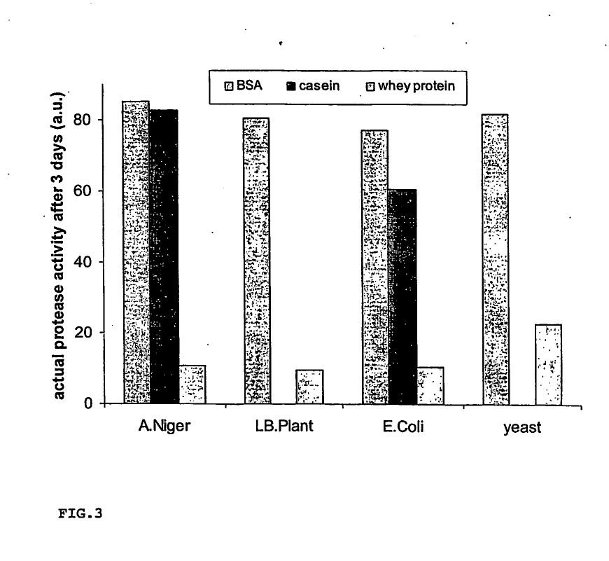

FIG. 3 shows the actual protease activity 3 days after contamination in a closed container, as it has been determined using a standard protease assay (Sigma, C0528). This has been done for different protein additions (0.1 wt. %) to the medium of Example 1.

On the horizontal axis of FIG. 3, the different test organisms are plotted (A. Niger: Aspergillus niger; LB. Plant: Lactobacillus plantarum; E. Coli: Escherichia coli; yeast: Saccharomyces cerevisiae), with, for each organism, the protease activity in reaction to three different additions in a bar chart. The left-bar represents BSA, the middle bar represents casein, and the right bar represents whey protein. On the vertical axis, the actual protease activity is plotted in arbitrary units (a.u.).

It appears from FIG. 3 that, preferably, BSA is used as a protein addition, since this results in the highest protease activity for all microorganisms tested.

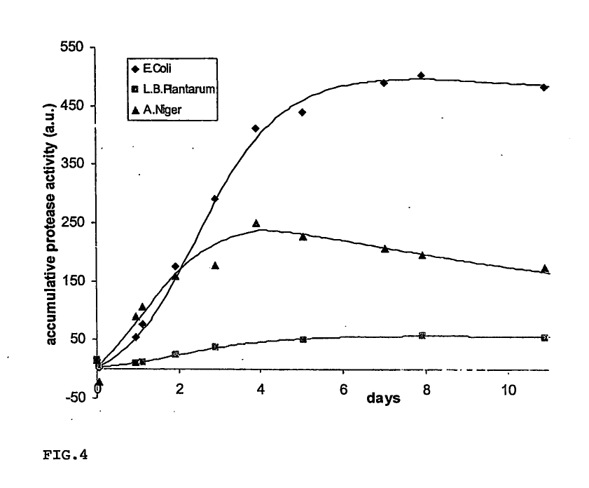

FIG. 4 shows the accumulative protease activity over a period of 11 days after contamination, as it has been determined using a fluorescein isothiocyanate-labeled casein (FITC casein) fluorescent indicator also present in the medium. Such an indicator becomes fluorescent after enzymatic hydrolysis of the casein by proteolytic enzymes, and can be manufactured by means of methods known to a person skilled in the art (e.g. Akopian et al. (1997) J. Biol. Chem. 272, 1791-8), but is also commercially available (Sigma; C0403). The experiment has been carried out for different types of contaminations (additions of test organisms) and with BSA (0.1 wt. %) as a protein addition to the medium of Example 1 in a closed container.

On the horizontal axis of FIG. 4, the time after contamination of the medium is plotted. On the vertical axis, the accumulative protease activity is plotted in arbitrary units (a.u.). The accumulative protease activity measured in time is plotted for three separate experiments carried out with the different test organisms Aspergillus niger (A. Niger, triangle), Lactobacillus plantarum (L. B. Plantarum, square) and Escherichia coli (E. Coli, diamond).

Also, similar measurements have been carried out with a sulforhodamine-labeled casein (sulforhodamine casein) fluorescent indicator for accumulative protease activity. This has yielded comparable results. In this manner, a non-invasive optical sensor is realized which can be attached on the inner side of the container wall.

It appears from FIG. 4 that the protease activity of the microorganisms tested increases over an initial growth period of the microorganisms, after which a stabilization of the fluorescence signal occurs, which means that the microorganisms have died and that, consequently, no proteases are produced anymore. So, the indicator or method used shows after 11 days that a contamination has taken place. An indicator or sensor by means of which the actual protease activity is measured, would incorrectly indicate after 11 days that there is no contamination (at that moment). For the intended purpose of the sterile (tissue) culture of plants, the accumulative indication method, in which an accumulative indicator is used and ended contaminations can also be observed, in addition to monitoring the contamination itself, whether or not in the form of living microorganisms, is considered an important advantage.

1. A method for detecting a contamination with a microorganism in a closed container, wherein an extracellular enzyme, or its activity, of said microorganism is detected.

2. A method according to claim 1, wherein the activity of said extracellular enzyme is detected by means of providing a substrate for said enzyme to the contents of the container and detecting the conversion of said substrate by said enzyme.

3. A method according to claim 2, wherein said detecting of the conversion comprises detecting the quantity of substrate and/or detecting a reaction product.

4. A method according to claim 1, wherein said enzyme, said substrate and/or said reaction product is detected by means of an indicator.

5. A method according to claim 1, wherein said indicator is located in a coating on the inner side of said container.

6. A method according to claim 1, wherein said microorganism is a plant cell, protozoon, fungus, yeast, archaeum or bacterium.

7. A method according to claim 1, wherein amylase, protease, lipase, pectinase, xylanase, cellulase, chitinase, collagenase and/or glucanase are detected.

8. A method according to claim 1, wherein said detecting comprises an optical measurement, preferably a non-invasive optical measurement.

9. A method according to claim 8, wherein the optical measurement comprises a fluorescent or (chemi)luminescent measurement.

10. A method according to claim 8, wherein an optical sensor is used.

11. A method according to claim 10, wherein said optical sensor comprises a chemo-optical substance.

12. A method according to claim 1, wherein said substrate comprises starch, protein, glyoxylate, substances with an aldehyde function, a carboxyl ester or an acetic acid ester, preferably protein.

13. A container for sterile tissue culture, which comprises an indicator for detecting an extracellular enzyme of a microorganism.
